(19) United States
(12) Patent Application Publication
(10) Pub. No.: US 2007/0111279 A1
(43) Pub. Date: May 17, 2007

(54) Production of recombinant therapeutic bioscavengers against chemical and biological agents

(75) Inventor: Yvonne Rosenberg, Washington, DC (US)

Correspondence Address: YVONNE ROSONBERG;APT 3, 1336 9th ST. NW, WASHINGTON, DC, 20001, US

(73) Assignee: Procell, Inc., Rockville, MD (US)

(21) Appl. No.: 11/188,044

(22) Filed: Jul. 25, 2005

(30) Foreign Application Priority Data
Nov. 13, 2002 (US) PCT/US02/36216

(51) Int. Cl. C12N 9/10; C07H 21/04; C12P 21/06; C12N 5/06

(52) U.S. Cl. 435/69.1; 536/23.2; 435/193; 435/358

(57) ABSTRACT

This application provides a method to produce recombinant bioscavenger molecules to be used as human treatments to protect against toxicity resulting from exposure to chemical/biological agent toxins or drugs. This invention relates to the production of glycoproteins that exhibit poor stability in vivo and are thus inadequate as therapeutic treatments without the additional post-translational modifications of the expressed molecules. In one embodiment, the method combines molecular and biochemical technologies; first for the expression of recombinant molecules and second for the in vitro glycoslyation of the purified or partially purified expressed molecules, intended to mimic the glycoprotein profiles of the native molecules. In another embodiment, post-translation modifications can be provided by direct genetic modification of the cells used in the protein expression system. Although the invention is intended for in vivo use, the invention allows for decontamination in vitro. The establishment of recombinant detoxification agents has applications in numerous terrorist, drug, and environmental scenarios involving military and civilian welfare.

### FIELD OF THE INVENTION

This disclosure relates to methods for developing a system for the production of recombinant proteins for use as an effective human antidote/anti-toxificant/anti-chemical warfare agent in vivo to prevent prolonged or toxic effects following chemical/biological exposure from toxins and drugs. In one embodiment, the disclosure relates to the production of functional bioscavenger cholinesterase molecules as a protective in vivo treatment against exposure to nerve agents and drugs including but not limited to cocaine, heroin and succinylcholine.

### BACKGROUND OF THE INVENTION

In general, both native proteins isolated from plasma and recombinant proteins produced using eukaryotic protein expression systems have been used as potential therapeutic human treatments in preclinical. While effective on a laboratory scale, purification from blood is time consuming and expensive, may suffer from batch to batch variability and may suffer from potential safety issues associated with contaminating infectious agents (HIV-1, hepatitis, prions, etc.). These factors highlight the importance of developing recombinant protein technology as an alternate strategy for the production of commercially important amounts of therapeutic glycoproteins.

The most common eukaryotic protein expression systems using molecular recombinant technologies to produce large quantities of specific therapeutic proteins are based on stable transfected Chinese hamster ovary (CHO) cells and recombinant baculovirus-infected insect cells. These systems are capable of scale up operations, yielding milligram of protein per liter of culture and have potential advantages over prokaryotic expression of foreign polypeptides, including: (i) eukaryotic post-translational modification of expressed protein(s), (ii) increased solubility of recombinant fusion proteins leading to increased protein yield, and (iii) production of large and/or complex polypeptides which are difficult to purify from prokaryotic cells. However, for some recombinant proteins additional modifications may be required to enhance or maintain certain desirable characteristics innate to the protein found in its natural biological source tissue (s). These modifications range from post-translational modifications including the addition of moieties to the recombinant protein for recognition and/or the addition of tags for detection and purification purposes, to the inclusion of proteins, portion of proteins, and/or peptides to facilitate their functional biological activity. These modifications need not be added post-translational but could be incorporated transcriptionally into the eukaryotic protein expression systems as fusion or co-expressed molecules, thereby incorporating their influence to the functional biological activity of the final protein during its formation

Of late, there is an increasing need to develop quick acting and efficient therapeutic bioscavenger/anti-toxicant molecules for defense against chemical and biological agents. Biocatalytic destruction of organophosphates using current treatment protocols is not optimal and more efficient technologies are being sought for safely protecting military and civilian personnel against chemical and biological weapons. Novel methods have been advanced using organophosphates hydrolyzing enzymes as potential bioscavengers. With the recognition that broad-spectrum organophosphorus insecticides and nerve agents are able to produce acute cholinergic effects by inhibition of acetylcholinesterase (ACHE) permitting continuous firing of neurons and the ability of acetylcholinesterase and butyrylcholinesterase (BChE) to rapidly detoxify the active components, research in mice, rats and monkeys has focused on the use of native cholinesterases as a mode of treatment to prevent organophosphates toxicity. Importantly, such enzymes have recently been shown to also effectively protect against cocaine and heroin overdose as well as succinylcholine-induced apnea.

A critical property for any prophylactic or therapeutic treatment, aimed at efficient detoxification, is good stability (or long retention times) following in vivo administration. However, unlike the relatively homogeneous native glycoprotein preparations which may consist of multimeric forms of complex bi-antennary types of glycan structures, recombinant butyrylcholinesterase forms commonly exhibit variation in the type of sugar residues found within each oligosaccharide which negatively impacts the rate of in vivo clearance from days or hours to minutes and reduces their usefulness as therapeutics. Several factors determine this microheterogeneity, alterations in the location and number of nonsialylated galactose and mannose residues and inefficient folding and tetramerization, the host cell type used for expression and its physiological status may influence post-protein glycan processing. Thus, until the problems with alterations in sialylation and monomers assembly are overcome, recombinant approaches to therapeutic detoxification of chemical and biological agents will not be a realistic prevention strategy against organophosphates poisoning.

Approaches to overcome these innate deficiencies have either involved exposing recombinant proteins in vitro to enzymes such as exoglycosidases and sialyltransferase or introducing liver-derived enzyme beta-galactoside alpha-2,6-sialyltransferase cDNA by gene transfer into those cells producing the recombinant protein. The in vitro incorporation of sialic acid into neuraminidase-treated recombinant proteins (developed specifically to allow effcient sialic acid capping of beta-galactose-exposed termini) has been shown to saturate >70% of the theoretical acceptor sites. Similarly, recombinant proteins produced by the gene-modified cells may display a higher proportion of fully sialylated glycans and more closely resemble native forms in both structure and pharmacokinetic behavior. Such findings are in agreement with data showing that liver (the in vivo source of many of these highly sialylated glycoproteins) contain sialyltransferase, involved in the sialylation of O-glycosidically linked carbohydrate chains on serum glycoproteins.

In addition to sialylation and post-translational protein modifications, another innate deficiency in our present eukaryotic protein expression systems is the ability to multimerize monomeric forms of expressed recombinant proteins. In most if not all cases expressed proteins require higher forms of structure to provide the in vivo stability to carry out their intended function. Multimerization of protein monomers into dimeric, trimeric, or tetrameric structures rely on protein-protein interactions. In some cases these interactions are intrinsic to the molecule; in others, cellular encoded proteins facilitate these oligomeric super-structures. There is an increasing awareness from a spectrum of genetic deficiencies that mutations in a linking or anchoring protein and not in the specific gene itself is responsible for various congenital syndromes. These deficiencies are caused by an uncoordinated expression of protein subunits and linking/anchoring proteins that normally determines the pattern of molecular forms, which in turn determines the localization and functionality of the resulting protein.

Thus, present eukaryotic protein expression systems may fail to produce effective therapeutic molecules due to at least two inappropriate post-translational modifications, that is, the level, location and nature of N-glycan capping and subunit assembly. Such processes are critical requirements for high level expression, antigenicity and the pharmacokinetic behavior of recombinant glycoproteins, thus defining their therapeutic capacity in vivo and pharmaceutical usefulness as potential human treatments.

### SUMMARY OF THE INVENTION

Disclosed herein is a method for producing functional recombinant glycoprotein enzymes with potent anti-toxicant properties to be used as effective prophylactic or therapeutic treatments for humans situated in environments high risk for chemical/biological agent or pesticide exposure. In one embodiment treatment is pre-exposure capable of prophylactic protection against potential exposure to chemical defense agents and pesticides. In another embodiment, treatment is provided post-treatment, following exposure to chemical/biological defense agents, pesticides or overdoses with drugs including but not limited to cocaine, heroin and succinylcholine. Protective treatment using commercially available amounts of the recombinant bioscavenger in a chemical form such that in vivo stability is similar to the native molecule, requires a new combination of molecular and biochemical technologies involving 1) optimized eukaryotic protein expression systems, 2) correction or completion of glycosylation by in vitro or intra-cell manipulation and 3) a safe and simple delivery mechanism.

The methods disclosed herein encompass a method or series of methods to accomplish the intended functional pharmaceutical role or roles of the recombinantly expressed protein through transcription, translation, and post-translational modifications. In one embodiment, the cDNA for a specific biological molecule or molecules that is introduced into cells capable of high-level protein expression by viral mediated delivery mechanisms including but not limited to murine leukemia virus (MuLV), adenovirus, adeno-associated virus (AAV), lentivirus, and canarypox vectors. The cells capable of high-level protein expression could be Chinese hamster ovary (CHO) cells or insect cells infected by recombinant baculoviruses, but are not limited to these protein expression systems. The protein expression system is preferably eukaryotic, but could be prokaryotic consisting of bacterial or yeast expression systems. In another embodiment, the nucleic acid for the biological agent could be transfected into the cells selected for protein expression, by chemical and/or mechanical means that bypasses the cellular membrane to gain access to the cellular chromatin structure where integration occurs.

The products are biological molecules that preferably bind and inactivate a chemical or biological toxin thereby preventing excessive damage to biological mammalian tissues. Although the agent is conceived to be a protein expressed using a eukaryotic protein expression system defined by a nucleic acid sequence(s), it need not be limited to such a form of the biological molecule, since, as a protein, it may require additional post-translational modifications to enable the agent to provide the necessary disabling function(s).

As a bioscavenger, receptor, or enzyme, a portion of the molecule may need to be removed or modified to make the protein soluble for example intracellular and transmembrane domains. As a said receptor or soluble receptor, the agent binds, sequesters, and clears the toxin as a complex from the body. As an enzyme, the agent binds, inactivates by enzymatic cleavage or non-enzymatic hydrolyzes resulting in metabolites that are no longer harmful to mammalian tissues and/or hasten the removal of the toxin from the host. Inactivation can occur, but is not limited to enzymatic cleavage, blocking of reactive moieties, masking of active site (s), sequestering to certain tissues, and/or clearance of the toxin as a bound or unbound complex.

A second aspect of the invention provides for the further post-translational correction or completion of the sialyated glycan moieties on the expressed glycoproteins so as to mimic the native glycosylation profile and to ensure in vivo stability and the long half-life required of a therapeutic scavenger. In one embodiment, in vitro post-modification of the recombinant protein product is achieved with a combination of enzymes, including, but not limited to glycosyl-transferases. In another embodiment, cells used in the protein expression system are modified to achieve native glycosylation patterns. Cell modification would be by gene transfer. Any number of viral or non-viral vectors or direct delivery methods could introduce any number of genes. Genes could code for enzymes capable of modifying proteins by the addition of carbohydrates, nucleic acids, lipids or other biological molecular moieties, examples are transferase, polymerases, but not limited to these functions. In addition, the introduced gene(s) could code for enzymes that are involved in organic molecule(s) or molecular group(s) transfer, examples are phosphosphorylase, methyltransferase; attachment proteins; and/or multimerization proteins or peptides. These said modifications are covered by this invention independent of said modifications being made by genetic manipulations to the cell itself and/or if said modifications were done in vitro to the recombinant protein following its synthesis. In vitro modifications are independent of whether the synthesized protein was pure (using any number of methods known in the art), partially purified, or present in crude supernatants collected from recombinant expressed protein cultures.

### In a Particular Embodiment, this Invention Describes

(a) A method for development of a mammalian expression system for the production of recombinant mammalian cholinesterases with chemical and functional characteristics similar to that of the native-derived form; a process to clone and express two forms of mammalian cholinesterase by the introduction of the cDNA into Chinese hamster ovary (CHO) cells by retroviral mediated transduction (EXAMPLE 1). One form will be the clone for the full-length native protein sequence, while the other form would code for a protein that fuses the mammalian cholinesterase to the Fc portion of human IgG1 (EXAMPLE 4). The cDNA would be for butyrylcholinesterase (EC3.1.1.8 acylcholine achydrolase pseudocholin-esterase, non-specific cholinesterase), a serine esterase (MW=345,000) comprised of four identical subunits containing 574 amino acids and held together by non-covalent bonds and contains 36 carbohydrate chains (23.9% by weight). However, a cDNA for acetylcholinesterase or variant forms of either cholinesterase or similar chelating enzyme could be used. In addition, it is presently envisioned that a peptide containing a proline rich attachment domain (PRAD) would be co-expressed with the cholinesterase gene. These cDNAs could be expressed in the same or separate retroviral vectors. The peptide encompasses the proline-rich attachment domain that is present in a collagenic tail subunit. The peptide is meant to serve the same function as the collagenic tail subunit, which is composed of three collagenic strands (ColQ), each attached to a tetramer of the cholinesterase catalytic subunit via a proline-rich attachment domain. The function of the collagenic tail subunit is to anchor the enzyme and is required for tetramerization of monomers (see EXAMPLE 2). The recombinant expressed cholinesterase will be affinity purified over procainamide columns, followed by in vitro sialylation and in vivo testing (see EXAMPLE 5).

(b) The recombinant protein will then undergo glycoprotein remodeling in vitro to restore or complete any incomplete sialylation and/or other determined glycosylation, including, but not limited to galactosylation and fucosylation (Example 4*). Purified glycosyl-transferases will be used in vitro to restore the post-translational modifications innate to the native protein, but missing from the recombinant protein.

(c) The invention is intended for in vivo use in any recipient requiring detoxification, although in vitro usage can also be envisioned. In one embodiment, delivery in vivo is via the intravenous or intramuscular routes by hypodermic. In another embodiment delivery is by an inhaler device, the number of puffs being determined by the weight of the individual, the pre-existing levels of scavenger and the LD50 of the toxic agent.

### DETAILED DESCRIPTION OF THE PREFERRED EMBODIMENTS

The present invention relates to the establishment of a process to produce, using molecular and biochemical techniques, therapeutic recombinant detoxifying agents that are capable of inactivating potential biohazardous molecules before they can cause biological tissue damage in a mammalian host. The invention is focused on the development of biologics for detoxification of harmful biologics and chemicals (including organophosphorus chemical agents) that contain carboxylic or phosphoric acid ester bonds. The process involves cloning of a biological molecule(s), introducing it into a protein expression system that is either engineered to express the required modification proteins/enzymes or to further expose in solution the recombinant protein to modification proteins/enzymes in order to reconstruct or enhance the native source glycosylation and biological profiles.

The present invention describes a method for the production and use of prophylactic and therapeutic recombinant glycoprotein treatments that are effective only with appropriate post-modification(s) required for in vivo stability and function. Applications include but are not limited to: (i) Protection against biological and chemical warfareagents in terrorist situations; (ii) Clinical treatment of drug overdosing with cocaine, heroin; (iii) Alleviating life threatening conditions such as succinylcholine-induced apnea; (iv) Insecticide/pesticide exposure of civilians. In addition to in vivo use, the invention could be incorporated into a product for decontamination of sensitive biological surfaces such as skin, immobilized to surface polymers or sponges for external detoxification and decontamination schemes, and/or incorporated into a product for biosensing. Pursuant of the present invention, recombinant cholinesterase was chosen as an example of a bioscavenger agent that could detoxify and protect against environmental and terrorist toxins. The invention is envisioned as a general way of constructing and expressing such functional molecules. The present invention is intended to generate large, consistent quantities of detoxifying agents displaying high stability upon long-term storage

Biological agents against which the present invention may be include, but not limited to bacteria, parasites, protozoa, fungi, prion, and viruses. Of the microorganisms of most concern are Bacillus anthracis (anthrax), Brucella melitensis, Francisella tularensis, botulinum toxin, and plague. Of the viruses of most concern are smallpox, other human poxvirus infections, hemorrhagic fever and poxvirus infections. The three viral hemorrhagic fever viruses dengue, hemorrhagic fever with renal syndrome, and Congo-Crimean hemorrhagic fever represent the diversity of potential hemorrhagic fever viruses that military forces may be exposed. Chemical agents against which the scavenging properties of present invention include, but not limited to nerve agents, vesicant agents, and blood agents. The chemicals of most concern include sulfur mustard (a pulmonary irritant gas phosgene), sarin, soman, VX and hydrogen cyanide gas.

In summary, the invention describes the production of stable and safe bioscavenger glycoproteins to be used as in vivo detoxification agents with a wide range of applications, including protection against lethal exposure to chemical and biological toxins or drugs. Such pre- or -post exposure treatments provide a method to protect biological tissue from damage from biological, chemical, or environmental toxins. Toxin inactivation is accomplished by modification of the protein expression system either by manipulation of the final synthesized product in vitro or by direct genetic manipulation of the cells. Independent of the stage (pre- or post-synthesis) of molecular modification(s) the goal of this invention is to bestow biological detoxification properties similar to or improved upon that found in natural biological systems. The invention is a biological agent expressed in state-of-the-art protein synthesis systems that allows the appropriate modification(s) of the cells used or the product made required to inactivate and remove harmful agents. Thus, disclosed are methods produce the therapeutic molecules to protect mammalian tissue from environmental insult that could lead to cellular death.

The following examples further illustrate experiments that have demonstrated reduction to practice and utility of selected preferred embodiments of the present invention, although they are in no way a limitation of the teachings or disclosure of the present invention as set forth herein.

### EXAMPLE 1

### Expression and Production of Butyrylcholinesterase in Chinese Hamster Ovary (CHO) Cells

Establishment of CHO cells that continuously produces and expresses primate (monkey or human) BChE demonstrates the principle of this invention. CHO cells were used that were stable transduced with a murine leukemia virus vector in which the BChE gene is driven by the long-terminal repeat regulatory region. The BChE expressed, which is predominantly monomeric, was tested to be biologically active. These cells were then adapted to grow in suspension in CHO-S-SFM (serum-free media). High cell densities, typically 2.0×10⁶ cells/ml were obtained from spinner flask cultures. Partial purification of BChE from CHO cell cultured media revealed that the level of impurities in SFM was significantly lower that the serum-supplemented DMEM. This suggests that additional steps need not be employed in the purification of butyrylcholinesterase from SFM. This would result in a reduction of the operating time by 50 h and boost the recovery yield of BChE to 75%.

### EXAMPLE 2

### Coexpression of a Peptide Together with Butyrylcholinesterase in CHO Cells Enhances Heteromeric Forms that Enhance Enzymatic Activity

The principle of this invention is further demonstrated by the ability to enhance tetramerization of expressed monomeric butyrylcholinesterase expressed in Chinese hamster ovary (CHO) cells by the co-expression of a proline-rich attachment domain as a peptide.

Data suggest that for optimal detoxification activity by BChE, the tetrameric form of the enzyme is required. A heteromeric form of AChE is found in mammalian skeletal muscle, formed by the attachment of the catalytic subunit to a triple helical collagen-like tail subunit. The function of the collagen-like tail is to anchor catalytic subunits to the basal lamina. The triple helical association of three collagen-like strands, ColQ, forms the tail. The proline-rich attachment domain (PRAD) of each strand can bind the catalytic subunit tetramer producing the asymmetric moieties.

To test the possibility that expression of the proline-rich attachment domain (PRAD) would facilitate and enhance higher order heteromeric association of BChE catalytic subunits and thus enzymatic activity, CHO cells will be transduced with two retroviruses, one containing the cDNA for butyrylcholinesterase, the another containing the nucleic acid sequence for a peptide coding for the PRAD domain.

### EXAMPLE 3

### Expression of a Tetrameric Mutant Butyrlcholinesterase in CHO Cells with Enhanced Scavenging Capability

The principle of this invention is further demonstrated by the ability to enhance the scavenging/antidote/neutralizing activity of the tetrameric butyrylcholinesterase expressed in Chinese hamster ovary (CHO) cells by site directed mutagenesis of the wild type gene including but not limited to the E197Q mutant.

In vitro data indicate that enhanced detoxification activity by tetrameric BChE (see example 2) can be achieved by generating mutant ChE molecules with site-specific mutations. The production of a therapeutic bioscavenger molecule with enhanced activity reduces the amount required in vivo for pre-or post-exposure treatment.

### EXAMPLE 4

### Genetic Expression of a Chimeric Protein Between the Butyrylcholinesterase Gene and the Fc Portion of Human Immunoglobulin IgG1

The principle of this invention could be further demonstrated by experiments using portions of common serum proteins to imprint circulatory properties to proteins that are not normally found naturally in the blood. The term properties refer to any characteristic that enhances the pharmacodynamic profile of the blood-borne butyrylcholinesterase.

A genetic construction on the nucleic acid level would be made by fusing the coding region of the BChE gene to the nucleic aid sequence coding for the Fc portion of the human immunoglobulin IgG1. This new gene would be constructed in such a way as to bestow properties intrinsic to the individual genes alone. The nucleic acid sequence coding for the in-frame fusion gene will be coded into a retroviral vector construction and transduced into CHO cells.

### EXAMPLE 5

### In Vitro Post-Translational Modification of Butyrylcholinesterase and/or Butyrylcholinesterase-Ig Fusion to Produce a Recombinant Protein with Properties Similar to the Native Form

The principle of this invention could be further demonstrated by experiments where the CHO recombinantly expressed BChE and BChE-Ig are treated with enzymes that either restore the glycosylation pattern that are found native to the naturally occurring enzyme or bestows properties that enhances the pharmacodynamic profile of a circulatory enzyme.

It has been demonstrated that stability of butyrylcholinesterase is associated with capping of the termal carbohydrate residues with sialic acid. Sialylation has been suggested to influence retention times of butyrlcholinesterase in the blood due to either an unknown tissue absorption or rapid clearance of the enzyme due to binding of uncapped galactose residue to receptors on hepatocytes.

The recombinant expressed protein will be subjected in vitro to enzymes that will allow efficient sialic acid capping of beta-galactose-exposed termini. This will be accomplished by exposure of the recombinant preparation before or after procainamide affinity column chromtography to axoglycosidases and sialyltransferase. The in vitro incorporation of sialic acid into neuraminidase-treated recombinant proteins has been shown to saturate most of the theoretical acceptor sites. By this process, the pharmacokinetic properties of the recombinant, enzyme will approach or equal those observed for the purified plasma-derived BChE.

### EXAMPLE 6

### Delivery of Recombinant Sialylated Butyrylcholinesterase and/or Butyrylcholinestearase-Ig Fusion into Mammalians as a Bioscavenger

In vivo experiments in mice, non-human primates, and ultimately clinical trials/treatments in humans could further demonstrate the principle of this invention.

Retention times of recombinant sialylated BChE will be made and compared to observed retention times of purified BChE from native serum source. Various routes of delivery will be explored including intravenous, oral, intraperitoneal, intramuscular via autoinjectors, and pulmonary/intranasal via puffer devices at different doses to establish optimal bioavailability and retention times. In addition to biowarfare, the recombinant preparation may be used to alleviate succinylcholine-induced apnea and to treat cocaine or other drug overdosed individuals.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertain and as may be applied to the essential features herein before set forth.

What is claimed is:

1. A method of construction of a post-translationally modified recombinant glycoprotein with properties that mimic a native bioscavenger molecule.

2. The genetic bioscavenger constructed molecule of claim 1 could be produced transiently or expressed permanently as a stable recombinant molecule in a protein expression system known in the art, but would preferably be produced in eukaryotic cells where appropriate post-translational modifications would be bestowed onto the expressed protein.

3. The preferred host genetic bioscavenger expressed protein of claim 2 could be any proliferating mammalian cell capable of genetic modification including, but not limited to Chinese Hamster Ovary (CHO) cells, mesenchymal (MSCs), and/or haemopoietic stem cells.

4. The introduction of the genetic bioscavenger coding sequence could be introduced in the said cells of claim 3 by either viral and/or non-viral methods.

5. The recombinant bioscavenger molecule of claim 1 encoded for one or more protein(s) belonging to a group of proteins known to bind, sequester, and/or inactivate any chemical or biological agent that is toxic toward mammalian cells. Such chemical nerve agents and insecticide antitoxicant proteins include, but not limited to, butyrylcholinesterase (BChE), acetylcholinesterase (AChE), organophosphate hydrolases (OPH), organophophorus acid anhydride hydrolases (OPAA), parathion hydrolase, paraoxonase and carboxylesterase.

6. The recombinant bioscavenger molecule of claim 2 encoded for a group of genes identified in claim 5 where mutations are introduced to improve their bioscavenging capability.

7. The recombinant wild-type (claim 5) or mutated (claim 6) bioscavenger molecule(s) binds, inactivates, and/or neutralizes chemical and/or biological toxins similar to the native protein of claim 1, preventing excessive damage to mammalian biological tissues and function as an effective antidote, anti-toxicant, and/or anti-chemical warfare agents in vivo.

8. The recombinant bioscavenger molecule of claim 7 can be used as both a pre-exposure (prophylactic) and/or post exposure treatment.

9. The cells that produce the recombinant bioscavenger molecule of claim 2 either contain or can be engineered to provide appropriate post-translational modification to mimic the glycosylation profiles of the native bioscavenger molecule.

10. Engineering cells in claim 9 could be accomplished by procedures identified in claim 4, resulting in the introduction of gene(s) encoding enzymes that are not expressed in the protein expression system used in claim 2. An example could be the addition of an enzyme-α2,6-sialyltransferase to CHO cells, but is not limited to this enzyme or cell type.

11. As an alterative to cellular modification, appropriate post-translational modifications can be performed after the protein is synthesized in a purified or non-purified preparation.

12. The in vitro method(s) to modify the recombinant bioscavenger protein of claim 2 could include, but not limited to glycosylation remodeling where the protein preparation in claim is incubated with appropriate enzymes in solution or coupled to a solid support. These enzymes include, but not limited to, glycosyltransferases such as sialtransferases, galactosyltransferases, and fucosyltransferases.

13. The in vitro modifications of claim 12 could include procedures that utilize the addition of biochemical precursors to the producer cell culture medium in order to optimize galactose capping or other modification or enhanced termination of desired glycosylation remodeling.

14. The construction of a recombinant bioscavenger molecule of claim 1 where the introduced gene(s) in question (claim 2) are included within a group that encodes for an antitoxicants against organophosphate nerve agents and pesticides and include but are not limited to butyrylcholinesterase (BChE), acetycholinesterase (AChE), organophosphate hydrolases (OPA), organophophorous acid anhydride hydrolases (OPAA), parathion hydrolase, paraoxonase and carboxyesterase.

15. The construction of a recombinant bioscavenger molecule of claim 1 where the introduced gene(s) in question (claim 2) are included within a group that encodes for antitoxicants against a drug(s) that include, but are not limited to, heroin, cocaine and apnea inducing succinyl choline.

16. The construction of a recombinant bioscavenger molecule of claim 4 where CHO cells in addition to the bioscavenger molecule produce a catalytic subunit that binds the proline-rich attachment domain at the C-terminal end of each cholinesterase (ChE) monomer, promoting the tetramerization of the relevant recombinant monomeric bioscavenger molecules.

17. The construction of a recombinant bioscavenger molecule of claim 8 wherein the amount of bioscavenger administered will protect against at least 0.5 LD50 depending on the nature and potency of the previous or anticipated nerve agent or insecticide exposure.

18. The construction of a recombinant bioscavenger molecule of claim 8 wherein the biological agents may include, but not limited to, bacteria, parasites, protozoa, fungi, prions, viruses, and/or toxins produced by the agents organism.

19. The construction of a recombinant bioscavenger molecule of claim 1 by a procedure where the post-translational modification produces glycosylation profiles mimicking that of the native molecule and enhancing in vivo stability.

20. The construction of a recombinant bioscavenger molecule that binds, inactivates, neutralizes chemical and/or biological toxins of claim 5 and that can be delivered by any route including intravenous, intramuscular, intraperitoneal (e.g.: using hypodermics), intrapulmonary (e.g.: using an inhaler), orally (e.g.: drinking/eating) and transcutaneously (e.g.: using a patch).
